(19) United States
(12) Patent Application Publication
(10) Pub. No.: US 2017/0081573 A1
(43) Pub. Date: Mar. 23, 2017

(54) A Discontinuous Silicone Adhesive Article

(71) Applicant: 3M INNOVATIVE PROPERTIES COMPANY, Saint Paul, MN (US)

(72) Inventors: Cary A. Kipke, Woodbury, MN (US); Wei Zhang, Woodbury, MN (US); Ronald W. Ausen, Saint Paul, MN (US)

(73) Assignee: 3M INNOVATIVE PROPERTIES COMPANY, Saint Paul, MN (US)

(21) Appl. No.: 15/312,096

(22) PCT Filed: May 15, 2015
(86) PCT No.: PCT/US2015/031078

§ 371 Date: Nov. 17, 2016

Related U.S. Application Data
(60) Provisional application No. 62/002,224, filed on May 23, 2014.

(51) Int. Cl. C09J 183/04; A61L 15/58; A61F 13/02; A61L 15/26; A61L 26/00; C09J 5/00; A61L 15/22

(52) U.S. Cl. CPC C09J 183/04; C09J 5/00; A61L 15/585; A61L 15/225; A61L 15/26; A61L 26/0061; A61L 26/0052; A61L 26/0019; A61F 13/0213; A61F 13/025; A61F 2013/00251

(57) ABSTRACT

A discontinuous silicone article is disclosed that includes a plurality of strands of radiation cured silicone gel arranged to form a net-like structure with openings between strands. The silicone gel provides adhesion to a surface, such as skin, and the openings provide for moisture transmission away from the surface. The discontinuous silicone article comprises at least one adhesive polymer strand and a plurality of joining strands. The adhesive polymer strands comprise a radiation cured silicone gel and each polymer strand repeatedly contacts an adjacent joining strand at bond regions.

### FIELD

The present disclosure relates to a discontinuous silicone adhesive article and a method of making a discontinuous silicone adhesive article.

### BACKGROUND

Silicone adhesives are useful for medical tapes and dressings because the silicone adhesive can provide adhesion to skin and gentle removal from skin without causing trauma or stripping skin cells or hair. The skin and especially a wound may produce moisture. Silicone adhesives are generally very hydrophobic and do not allow for fluid absorption or fluid passage. Therefore, moisture from the skin can weaken the adhesive bonding to skin and cause the adhesive to lift from the skin. Also, moisture from the skin can become trapped and possibly cause skin maceration.

To help with removing moisture or fluid from the skin, a hydrophilic silicone could be blended with a hydrophobic silicone to improve moisture absorption, see for example U.S. Pat. No. 7,842,752. In other designs, incorporation of absorbent particles into a hydrophobic adhesive can help with increasing absorbency. However, for either, the ability of the adhesive system to absorb water is limited.

Including through-holes in the silicone adhesive layer can help with fluid management. For example, U.S. Pat. No. 5,540,922, discloses a silicone adhesive on a supporting film, wherein the silicone adhesive and supporting film are perforated to allow for fluid passage. However, perforating the adhesive coated film results in wasted material during the production process and therefore increases cost. In addition, a perforation process increases the risk that particles or debris created from the cutting process becoming embedded into the silicone adhesive and introduced to the skin or wound.

### SUMMARY

A discontinuous silicone article is disclosed that includes a plurality of strands of radiation cured silicone gel arranged to form a net-like structure with openings between strands. The silicone gel provides adhesion to a surface, such as skin, and the openings provide for moisture transmission away from the surface.

In one embodiment, the discontinuous silicone article comprises at least one adhesive polymer strand and a plurality of joining strands. The adhesive polymer strands comprise a radiation cured silicone gel and each polymer strand repeatedly contacts an adjacent joining strand at bond regions.

In one embodiment, the silicone gel comprises a crosslinked poly diorganosiloxane material. In one embodiment, the poly diorganosiloxane material comprises a poly dimethylsiloxane. In one embodiment, the poly dimethylsiloxane is selected from the group consisting of one or more silanol terminated poly dimethylsiloxanes, one or more non-functional poly dimethylsiloxanes, and combinations thereof. In one embodiment, the poly dimethylsiloxane consists of one or more non-functional poly dimethylsiloxanes. In one embodiment, the adhesive polymer strands further comprise a silicate resin tackifier. In one embodiment, the adhesive polymer further comprises a poly(dimethylsiloxane-oxamide) linear copolymer. In one embodiment, the crosslinked poly diorganosiloxane material comprises a crosslinked poly dimethylsiloxane material and the noncrosslinked poly diorganosiloxane fluid comprises a noncrosslinked poly dimethylsiloxane fluid. In one embodiment, the poly diorganosiloxane material comprises a poly diorganosiloxane fluid having a dynamic viscosity at 25° C. of no greater than 500,000 mPa·sec. In one embodiment, the poly diorganosiloxane material comprises a poly diorganosiloxane fluid having a dynamic viscosity at 25° C. of no greater than 100,000 mPa·sec. In one embodiment, the adhesive polymer further comprises a hydrophilic polymer.

In one embodiment, the polymer strands and joining strands do not substantially cross over each other. In one embodiment, the polymer strand is adjacent to a first joining strand and a second joining strand. In one embodiment, a plurality of first bond regions form between the polymer strand and the first joining strand each spaced from one another. In one embodiment, a plurality of second bond regions form between the polymer strand and the second joining strand each spaced from one another. In one embodiment, the joining strands each form a substantially straight line. In one embodiment, the plurality of polymer strands each form a wave. In one embodiment, an opening is formed between the polymer strand and the first joining strand in an area between the successive first bonding regions. In one embodiment, an opening formed between the polymer strand and the second joining strand in an area between the successive second bonding regions. In one embodiment, the openings form at least 25% of the area of the discontinuous silicone article.

In one embodiment, the joining strands comprise a thermoplastic resin, an elastomeric material, an adhesive, a hydrophobic polymer, or a release material. In one embodiment, the joining strands are the same composition as the polymer strands. In one embodiment, the article further comprises a backing to which the plurality of polymer strands and joining strands are secured. In one embodiment, the backing is a woven, knitted, nonwoven, film, paper, foam. In one embodiment, the backing is coated with adhesive. In one embodiment, the backing extends beyond the polymer strands and joining strands.

In one embodiment, the discontinuous silicone article comprises a plurality of joining strands and an plurality of adhesive polymer strands, wherein the adhesive polymer strands are formed by exposing a composition comprising a poly diorganosiloxane material to at least one of electron beam irradiation and gamma irradiation at a sufficient dose to crosslink the poly diorganosiloxane material and form a radiation cured silicone gel, wherein the silicone gel comprises a crosslinked poly diorganosiloxane material and a poly(dimethylsiloxane-oxamide) linear copolymer, a plurality of joining strands. Each polymer strand repeatedly contacts an adjacent joining strand at bond regions.

In one embodiment, the method of making the silicone article further comprises dispensing through a first orifice at a first speed a polymer strand, which comprises silicone material, dispensing through a second orifice at a second speed a first joining strand on a first side of the polymer strand, wherein the first speed is faster than the second speed, dispensing through a third orifice at the second speed a second joining strand on a second side of the polymer strand, opposite the first side, applying radiation to the silicone material to cure the silicone material to form a silicone gel. In one embodiment, the method of making the silicone article comprises oscillating the polymer strand between the first joining strand to form a first bond region and the second joining strand to form a second bond region. In one embodiment, the joining strands each form a substantially straight line. In one embodiment, the method of making the silicone article further comprises oscillating the first joining strand, oscillating the second joining strand, oscillating the polymer strand.

In one embodiment, the method of making the silicone article further comprises forming an opening between the polymer strand and the first joining strand in an area between the successive first bonding regions. In one embodiment, the method of making the silicone article further comprises forming an opening between the polymer strand and the second joining strand in an area between the successive second bonding regions.

In one embodiment, the method of making the silicone article further comprises applying e-beam radiation to the silicone material to cure the silicone material to form a silicone gel. In one embodiment, the method of making the silicone article further comprises applying e-beam radiation within 10 minutes of dispensing of the silicone material to cure the silicone material to form a silicone gel.

In one embodiment, the method of making the silicone article further comprises heating the silicone material to extrude through the first orifice at a first speed. In one embodiment, the method of making the silicone article further comprises heating the silicone material of the polymer strand to extrude through the first orifice, heating the material of the first joining strand to extrude through the second orifice, and heating the material of the second joining strand to extrude through the third orifice.

The word “strand” as used herein means an elongated filament.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments.

As used herein, “a,” “an,” “the,” “at least one,” and “one or more” are used interchangeably. The term “and/or” (if used) means one or all of the identified elements or a combination of any two or more of the identified elements.

### BRIEF DESCRIPTION OF THE DRAWINGS

FIG. 1 is a perspective view of a first embodiment of a discontinuous silicone article;

FIG. 2 is a perspective view of a second embodiment of a discontinuous silicone article;

FIG. 3 is a top view of a medical dressing with the discontinuous silicone article of FIG. 1;

FIG. 4 is a perspective view of a dispensing die for dispensing strands;

FIG. 5 is a side view of a portion of the process for dispensing strands for making the discontinuous silicone article.

While the above-identified drawings and figures set forth embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of this invention. The figures may not be drawn to scale.

### DETAILED DESCRIPTION

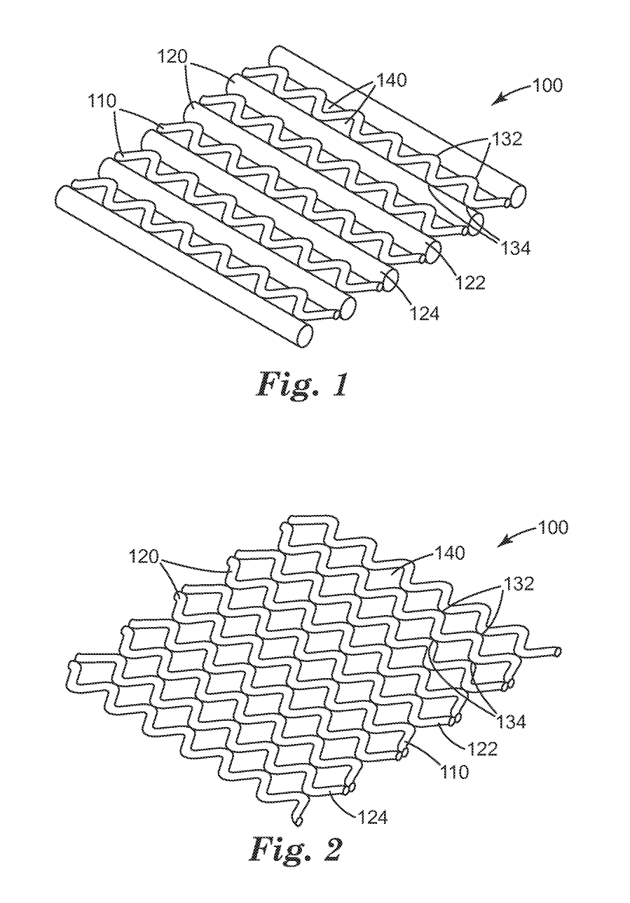

FIG. 1 is a perspective view of a first embodiment and FIG. 2 is a perspective view of a second embodiment, each showing a discontinuous silicone article 100, which comprises a plurality of polymer strands 110 and joining strands 120. A polymer strand 110 repeatedly contacts an adjacent first joining strand 122 at a various first bond regions 132, which are each successively spaced from one another. The polymer strand 110 repeatedly contacts an adjacent second joining strand 124 at various second bond regions 134, which are each successively spaced from one another. The spacing between successive first bond regions 132, and between successive second bond regions 134 forms openings 140. The openings 140 are essentially free of substance. In one embodiment, such as shown in FIGS. 1 and 2, the polymer strands 110 and joining strands 120 do not substantially cross over each other. In one embodiment, the discontinuous silicone article 100 has a net-like structure.

In one embodiment, the openings 140 form at least 5% of the area of the silicone article 100. In one embodiment, the openings 140 form at least 10% of the area of the silicone article 100. In one embodiment, the openings 140 form at least 25% of the area of the silicone article 100. In one embodiment, the openings 140 form less than 60% of the area of the silicone article 100. In one embodiment, the openings 140 form less than 40% of the area of the silicone article 100.

In one embodiment, the polymer strands 110 have a cross section wherein the strand 110 is widest in the middle portion and narrower at the upper and lower portion. For example, in one embodiment, the polymer strands 110 have a circular cross section. In contrast, perforated structures would have a cross section with side walls in a straight line. At each opening 140 the size of each opening 140 is larger at the surfaces of the article 100 than in the middle of the article 100. In other words, at a cross section an opening 140 is widest at the bottom and again at the top.

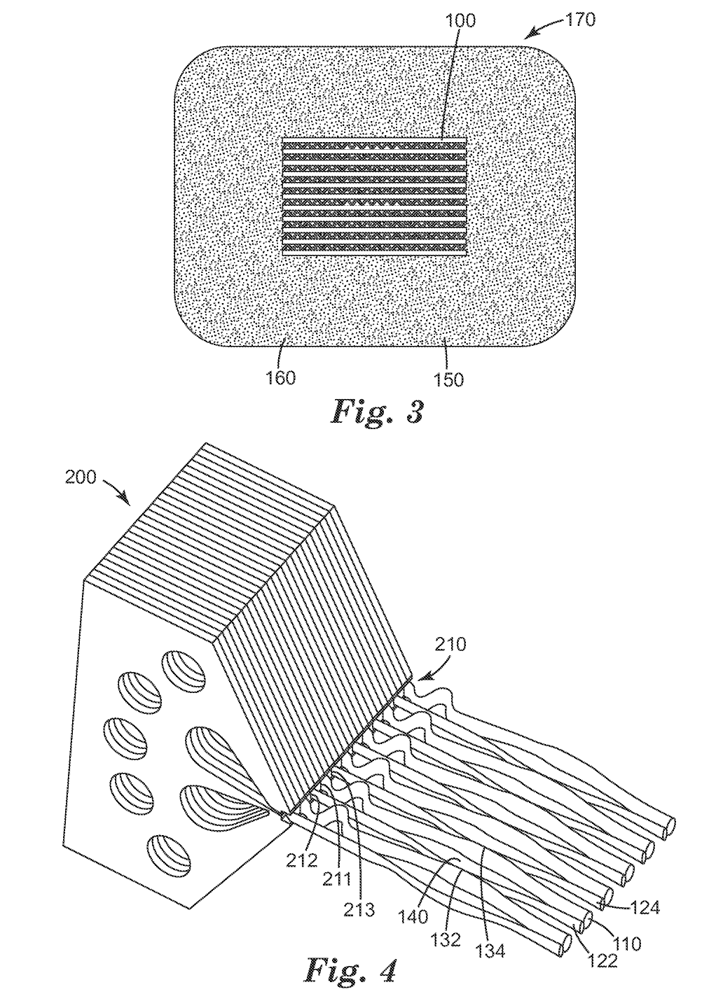

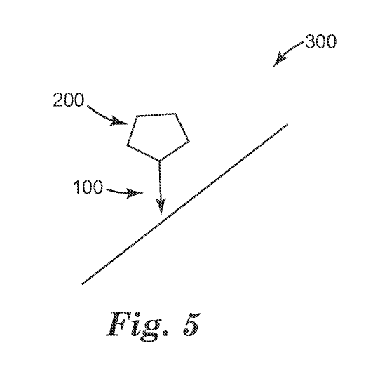

The polymer strands 110 are continuous along an x-axis, and the joining strands 120 are continuous along an x-axis (see FIGS. 1 and 2). The plurality of first bond regions 132 between the polymer strand 110 and the first joining strand 122, along with the plurality of second bond regions 134 between the polymer strand 110 and the second joining strand 124 result in the silicone article 100 having a structure that creates a barrier in the y-axis as well as the x-axis. Limiting fluid flow along both an x-axis and y-axis is beneficial for when the silicone article 100 (with a backing 150 applied to limit z-axis flow as well, see FIG. 3) is used on skin to limit external contaminants from entering into the covered area and to limit wound fluid from exiting the covered area.

In the embodiment of FIG. 1, the joining strands 120 are each formed in substantially straight lines, while the polymer strands 110 undulate between adjacent joining strands 120 and form a wave-like line. In the embodiment of FIG. 2, the joining strands 120 and the polymer strands 110 each undulate to form a wave-like line.

Various width, dimensions, amplitude and frequency of wave for each polymer strand 110 or joining strand 120 could be used so long as the polymer strand 110 repeatedly contacts an adjacent joining strand 120, and so long as openings 140 form between bond regions 132, 134.

In some embodiments, the silicone article 100 has a thickness greater than 0.025 mm. In one embodiment, the silicone article 100 has a thickness less than 2.54 mm.

In some embodiments, the polymer strands 110 have an average width in a range from 10 micrometers to 500 micrometers (in a range from 10 micrometers to 400 micrometers, or even 10 micrometers to 250 micrometers). In some embodiments, the joining strands 120 are of the same size as the polymer strands 110. In some embodiment, the joining strands 120 are smaller or larger than the polymer strands 110.

In some embodiments, silicone article 100 has a basis weight in a range from 5 g/m² to 2000 g/m² (in some embodiments, 10 g/m² to 400 g/m²).

The joining strand 120 may comprise a thermoplastic resin, an elastomeric material, an adhesive, a silicone gel, a release material, or any composition of strand such as disclosed in WO 2013/032683, so long as the joining strand 120 is of a composition that is capable of bonding with polymer strand(s). In one embodiment, the joining strand 120 is radiation cured. In one embodiment, the joining strand 120 is a radiation cured silicone gel. In one embodiment, the joining strand 120 is of the same composition as the polymer strand 110.

For the discontinuous silicone adhesive article of the present disclosure, at least one polymer strand 110 comprise a radiation cured silicone gel. In one embodiment, all polymer strands 110 comprise a radiation cured silicone gel.

Silicone gel (crosslinked poly dimethylsiloxane (“PDMS”) materials have been used for dielectric fillers, vibration dampers, and medical therapies for promoting scar tissue healing. Lightly crosslinked silicone gels are soft, tacky, elastic materials that have low to moderate adhesive strength compared to traditional, tackified silicone PSAs. Silicone gels are typically softer than silicone pressure sensitive adhesives (“PSA”), resulting in less discomfort when adhered to skin. The combination of relatively low adhesive strength and moderate tack make silicone gels suitable for gentle to skin adhesive applications.

Silicone gel adhesives provide good adhesion to skin with gentle removal force and have the ability to be repositioned. Examples of commercially available silicone gel adhesive systems include products marketed with the trade names: Dow Corning MG 7-9850, WACKER 2130, BLUESTAR 4317 and 4320, and NUSIL 6345 and 6350.

These silicone gel adhesives are formed by an addition cure reaction between vinyl-terminated poly(dimethylsiloxane) (PDMS) and hydrogen terminated PDMS, in the presence of a hydrosilation catalyst (e.g., platinum complex). Vinyl-terminated and hydrogen terminated PDMS chains are referred to as ‘functionalized’ silicones due to their specific chemical moieties. Individually, such functional silicones are generally not reactive; however, together they form a reactive silicone system. Additionally, silicate resins (tackifiers) and PDMS with multiple hydrogen functionalities (crosslinkers) can be formulated to modify the adhesive properties of the gel.

The silicone gel adhesives resulting from the addition cure reaction are very lightly crosslinked polydimethysiloxane (PDMS) networks with some level of free (not crosslinked) PDMS fluid and little or no tackifying resin. By contrast, tackifying resins are typically used at high levels (45-60 pph) in silicone PSAs.

In addition to the catalyst-promoted curing of silicone materials, it is known that free radicals formed from the high temperature degradation of organic peroxides can crosslink or cure silicone formulations. This curing technique is undesirable due to the acidic residues left in the film from the curing chemistry, which are corrosive and unsuitable for skin contact. In addition, this curing technique is too slow to cross-link the silicone material in sufficient time to maintain the openings 140 of the discontinuous article 100.

Generally, the crosslinked siloxane networks of the present disclosure can be formed from either functional or non-functional silicone materials. These gel adhesives have excellent wetting characteristics, due to the very low glass transition temperature (Tg) and modulus of the polysiloxane network. Rheologically, these gels exhibit nearly identical storage moduli at bond making and bond breaking time scales, resulting in relatively low to moderate forces being required to debond the adhesive by peeling. This results in minimal to no skin trauma upon removal. Additionally, the elastic nature of the crosslinked gel prevents flow of the adhesive around hair during skin wear, further reducing the instances of pain during removal.

Generally, the silicone materials may be oils, fluids, gums, elastomers, or resins, e.g., friable solid resins. Generally, lower molecular weight, lower viscosity materials are referred to as fluids or oils, while higher molecular weight, higher viscosity materials are referred to as gums; however, there is no sharp distinction between these terms. Elastomers and resins have even higher molecular weights than gums, and typically do not flow. As used herein, the terms “fluid” and “oil” refer to materials having a dynamic viscosity at 25° C. of no greater than 1,000,000 mPa·sec (e.g., less than 600,000 mPa·sec), while materials having a dynamic viscosity at 25° C. of greater than 1,000,000 mPa·sec (e.g., at least 10,000,000 mPa·sec) are referred to as “gums”.

Generally, the silicone materials useful in the present disclosure are poly diorganosiloxanes, i.e., materials comprising a polysiloxane backbone. In some embodiments, the poly diorganosiloxane is a homopolymer, containing no functional silicone segments or copolymers. In some embodiments, the nonfunctionalized silicone materials can be a linear material described by the following formula illustrating a siloxane backbone with aliphatic and/or aromatic substituents:

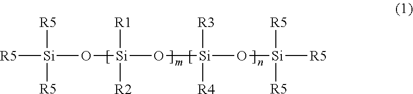

wherein R1, R2, R3, and R4 are independently selected from the group consisting of an alkyl group and an aryl group, each R5 is an alkyl group and n and m are integers, and at least one of m or n is not zero. In some embodiments, one or more of the alkyl or aryl groups may contain a halogen substituent, e.g., fluorine. For example, in some embodiments, one or more of the alkyl groups may be —CH₂CH₂C₄F₉.

In some embodiments, R5 is a methyl group, i.e., the nonfunctionalized poly diorganosiloxane material is terminated by trimethylsiloxy groups. In some embodiments, R1 and R2 are alkyl groups and n is zero, i.e., the material is a poly(dialkylsiloxane). In some embodiments, the alkyl group is a methyl group, i.e., poly(dimethylsiloxane) (“PDMS”). In some embodiments, R1 is an alkyl group, R2 is an aryl group, and n is zero, i.e., the material is a poly(alkylarylsiloxane). In some embodiments, R1 is methyl group and R2 is a phenyl group, i.e., the material is poly(methylphenylsiloxane). In some embodiments, R1 and R2 are alkyl groups and R3 and R4 are aryl groups, i.e., the material is a poly(dialkyldiarylsiloxane). In some embodiments, R1 and R2 are methyl groups, and R3 and R4 are phenyl groups, i.e., the material is poly(dimethyldiphenylsiloxane).

In some embodiments, the nonfunctionalized poly diorganosiloxane materials may be branched. For example, one or more of the R1, R2, R3, and/or R4 groups may be a linear or branched siloxane with alkyl or aryl (including halogenated alkyl or aryl) substituents and terminal R5 groups.

As used herein, “nonfunctional groups” are either alkyl or aryl groups consisting of carbon, hydrogen, and in some embodiments, halogen (e.g., fluorine) atoms. As used herein, a “nonfunctionalized poly diorganosiloxane material” is one in which the R1, R2, R3, R4, and R5 groups are nonfunctional groups.

Generally, functional silicone systems include specific reactive groups attached to the polysiloxane backbone of the starting material (for example, hydrogen, hydroxyl, vinyl, allyl, or acrylic groups). As used herein, a “functionalized poly diorganosiloxane material” is one in which at least one of the R-groups of Formula 2 is a functional group.

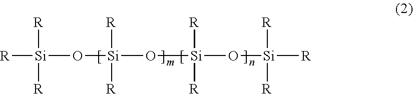

In some embodiments, a functional poly diorganosiloxane material is one in which at least 2 of the R-groups are functional groups utilized for cross-linking. Generally, the R-groups of Formula 2 may be independently selected. In some embodiments, at least one functional group utilized for cross-linking is selected from the group consisting of a hydride group, a hydroxy group, an alkoxy group, a vinyl group, an epoxy group, and an acrylate group.

In addition to functional R-groups, the R-groups may be nonfunctional groups, e.g., alkyl or aryl groups, including halogenated (e.g., fluorinated) alky and aryl groups. In some embodiments, the functionalized poly diorganosiloxane materials may be branched. For example, one or more of the R groups may be a linear or branched siloxane with functional and/or non-functional substituents.

The adhesives of the present disclosure may be prepared by combining one or more poly diorganosiloxane materials (e.g., silicone oils or fluids), optionally with an appropriate tackifying resin, dispensing it through a die to form the polymer strand 110 and optionally joining strand 120, and radiation curing using, for example, electron beam (E-beam) or gamma irradiation. Generally, any known additives useful in the formulation of adhesives may also be included.

If included, generally, any known tackifying resin may be used, e.g., in some embodiments, silicate tackifying resins may be used. In some exemplary adhesive compositions, a plurality of silicate tackifying resins can be used to achieve desired performance. The amount of tackifying resin in the silicone gel adhesive may be up to 10%, 20%, 30%, 40%, or 50% (wt.).

Suitable silicate tackifying resins include those resins composed of the following structural units M (i.e., monovalent R′₃SiO_{1/2} units), D (i.e., divalent R′₂SiO_{2/2} units), T (i.e., trivalent R′SiO_{3/2} units), and Q (i.e., quaternary SiO_{4/2} units), and combinations thereof. Typical exemplary silicate resins include MQ silicate tackifying resins, MQD silicate tackifying resins, and MQT silicate tackifying resins. These silicate tackifying resins usually have a number average molecular weight in the range of 100 to 50,000 gm/mole, e.g., 500 to 15,000 gm/mole and generally R′ groups are methyl groups.

MQ silicate tackifying resins are copolymeric resins where each M unit is bonded to a Q unit, and each Q unit is bonded to at least one other Q unit. Some of the Q units are bonded to only other Q units. However, some Q units are bonded to hydroxyl radicals resulting in HOSiO_{3/2} units (i.e., “T^{OH}” units), thereby accounting for some silicon-bonded hydroxyl content of the silicate tackifying resin.

The level of silicon bonded hydroxyl groups (i.e., silanol) on the MQ resin may be reduced to no greater than 1.5 weight percent, no greater than 1.2 weight percent, no greater than 1.0 weight percent, or no greater than 0.8 weight percent based on the weight of the silicate tackifying resin. This may be accomplished, for example, by reacting hexamethyldisilazane with the silicate tackifying resin. Such a reaction may be catalyzed, for example, with trifluoroacetic acid. Alternatively, trimethylchlorosilane or trimethylsilylacetamide may be reacted with the silicate tackifying resin, a catalyst not being necessary in this case.

MQD silicone tackifying resins are terpolymers having M, Q and D units. In some embodiments, some of the methyl R′ groups of the D units can be replaced with vinyl (CH2═CH—) groups (“D^{Vi}” units). MQT silicate tackifying resins are terpolymers having M, Q and T units.

Suitable silicate tackifying resins are commercially available from sources such as Dow Corning (e.g., DC 2-7066), Momentive Performance Materials (e.g., SR545 and SR1000), and Wacker Chemie AG (e.g., BELSIL TMS-803).

In some embodiments, the adhesives may include any of a variety of known fillers and additives including, but not limited to, tackifiers (e.g., MQ resins), fillers pigments, additives for improving adhesion, additives for improving moisture-vapor transmission rate, pharmaceutical agents, cosmetic agents, natural extracts, silicone waxes, silicone polyethers, hydrophilic polymers and rheology modifiers. Additives used to improve adhesion, particularly to wet surfaces, include polymers such as poly(ethylene oxide) polymers, poly(propylene oxide) polymers and copolymers of poly(ethylene oxide and propylene oxide), acrylic acid polymers, hydroxyethyl cellulose polymers, silicone polyether copolymers, such as copolymers of poly(ethylene oxide) and polydiorganosiloxane and copolymers of poly(propylene oxide) and polydiorganosiloxane, and blends thereof. The silicone polymer matrix may further comprise an absorbent particle or fiber dispersed. For example, PCT Publication WO2013/025955, the disclosure of which is herein incorporated by reference, discloses a silicone adhesive composition suitable for use in the polymer and/or joining strand.

The polysiloxane material, the tackifying resin, if present, and any optional additives may be combined by any of a wide variety of known means prior to being coated and cured. For example, in some embodiments, the various components may be pre-blended using common equipment such as mixers, blenders, mills, extruders, and the like.

In some embodiments, the materials may be dissolved in a solvent, dispensed through a die, and dried prior to curing. In some embodiments, solventless compounding and dispensing through a die may be used. In some embodiments, solventless dispensing through a die may occur at about room temperature. For example, in some embodiments, the materials may have kinematic viscosity of no greater than 100,000 centistokes (cSt), e.g., no greater than 50,000 cSt. However, in some embodiments, hot melt processing such as extrusion may be used, e.g., to reduce the viscosity of higher molecular weight materials. The various components may be added together, in various combinations or individually, through one or more separate ports of an extruder, blended (e.g., melt mixed) within the extruder, and extruded to form the hot melt composition.

The discontinuous silicone article 100 disclosed herein can be made by a process referred to a profile extrusion. For example, publication WO 2013/032683, the disclosure of which is herein incorporated by reference, discloses a profile extrusion process suitable for making the disclosed discontinuous silicone article 100. FIG. 4 shows a perspective view of an exemplary die 200 for dispensing material for making the polymer and joining strands 110, 120, respectively.

For materials of relative low viscosity at room temperature (i.e., a dynamic viscosity at 25° C. of no greater than 1,000,000 mPa·sec.), it is not necessary to heat the materials prior to sending through the die 200 for forming the polymer and joining strands 110, 120, respectively. Instead, these low viscosity materials can be dispensed through the die 200 at room temperature through gravity. In some embodiments, pressure from a pump can be used to dispense through the die 200. In some embodiments, heat can be used to dispense material through the die 200.

Generally, the profile extrusion process comprises die 200 including a plurality of orifices 210 for dispensing the polymer strands 110 and joining strands 120, which are spaced from one another. In general, it has been observed that the rate of strand bonding is proportional to the dispensing speed of the faster strand. Dispenser speed, orifice size, composition properties, for example, can be used to control the speed of the dispensed polymer strand and joining strands.

In one embodiment, the spacing between orifices is greater than the resultant diameter of the strand after being dispensed through the die, which leads to the strands repeatedly colliding with each other to form the bond regions. If the spacing between orifices is too great the strands will not collide with each other and will not form the bond regions. Typically, the polymer strands are dispensed in the direction of gravity. This enables collinear strands to collide with each other before becoming out of alignment with each other. In some embodiments, it is desirable to dispense the strands horizontally, especially when the extrusion orifices of the first and second polymer are not collinear with each other.

In one embodiment, the polymer strand 110 is dispensed from a first orifice 211 at a first speed, while a first joining strand 122 on a first side of the polymer strand 110 from a second orifice 212 and a second joining strand 124 on a second side of the polymer strand 110, opposite the first side, from a third orifice 213 both at the second speed.

In one embodiment, the extruded polymer strand 110, first joining strand 122, and second joining strand 124 do not substantially cross over each other. In one embodiment, the polymer strand 110 is oscillated between the first joining strand 122 to form the first bond region 132 and the second joining strand 124 to form the second bond region 134. Opening 140 is formed between the polymer strand 110 and the first joining strand 122 in the area between the successive first bonding regions 132 and is formed between the polymer strand 110 and the second joining strand 124 in the area between then successive second bonding regions 134.

In one embodiment, the joining strands 122, 124 each form a substantially straight line. In one embodiment, both polymer strands 110 and joining strands 122, 124 oscillate.

Typically, the orifice of the die is relatively small. In one embodiment, the orifice is less than 50 mil (1270 micron), and in one embodiment less than 30 mil (762 micron).

Regardless of how it is formed, the polymer strands 110 are radiation cured. If the joining strands 120 are also a silicone material, they are also radiation cured. In some embodiments, the strands may be cured through exposure to irradiation, such as e-beam irradiation. In some embodiments, the strands may be cured through exposure to gamma irradiation. In some embodiments, a combination of electron beam curing and gamma ray curing may be used. For example, in some embodiments, the strands may be partially cured by exposure to electron beam irradiation. Subsequently, the strands may be further cured by gamma irradiation.

A variety of procedures for E-beam and gamma ray curing are well-known. The cure depends on the specific equipment used, and those skilled in the art can define a dose calibration model for the specific equipment, geometry, and line speed, as well as other well understood process parameters.

Commercially available electron beam generating equipment is readily available. For examples, the radiation processing may be performed on a Model CB-300 electron beam generating apparatus (available from Energy Sciences, Inc. (Wilmington, Mass.)). Generally, a support film (e.g., polyester terephthalate support film) runs through a chamber. In some embodiments, a sample of uncured material with a liner (e.g., a fluorosilicone release liner) on both sides (“closed face”) may be attached to the support film and conveyed at a fixed speed of about 6.1 meters/min (20 feet/min). In some embodiments, a sample of the uncured material may be applied to one liner, with no liner on the opposite surface (“open face”). Generally, the chamber is inerted (e.g., the oxygen-containing room air is replaced with an inert gas, e.g., nitrogen) while the samples are e-beam cured, particularly when open-face curing.

The uncured material may be exposed to E-beam irradiation from one side through the release liner. For making a single layer laminating adhesive type tape, a single pass through the electron beam may be sufficient. Thicker samples, may exhibit a cure gradient through the cross section of the adhesive so that it may be desirable to expose the uncured material to electron beam radiation from both sides.

Commercially available gamma irradiation equipment includes equipment often used for gamma irradiation sterilization of products for medical applications. In some embodiments, such equipment may be used to cure, or partially cure the strands of the present disclosure. In some embodiments, such curing may occur simultaneously with a sterilization process for a semi-finished or finished product, for example a tape or wound dressing.

For embodiment of the uncured polymer and joining strands 110, 120, respectively that are flowable at room temperature, it is desirable to cure the material quickly after dispensing from the die 200 to preserve the discrete shape of the strands, open areas, and bond regions. In one embodiment, the discontinuous silicone article 100 is radiation cured within 10 minutes of being dispensed from the die 200. In one embodiment, the discontinuous silicone article 100 is radiation cured within 2 minutes of being dispensed from the die 200. In one embodiment, the discontinuous silicone article 100 is radiation cured within 10 seconds of being dispensed from the die 200.

In one embodiment, an additional backing 150 is included on a side of the discontinuous silicone article 100. The backing 150 may be a single or multilayer structure. In some embodiments, a backing that is transparent is desirable to allow for viewing of the underlying skin or medical device. The backing 150 may comprise fabric (such as woven, knitted, nonwoven), paper, film, foam, and combinations thereof. The backing 150 may include an adhesive 160 coating to aid in securing the silicone article 100 to the backing 150. In some embodiments, the backing 150 coincides in overall size with the silicone article 100. In some embodiment, the backing 150 extends beyond the overall size of the silicone article 100, and the adhesive 160 can be further used to aid in securing to the underlying surface or skin. The silicone article 100 may be applied directly to the backing and secure without including an additional adhesive.

In one embodiment, the backing 150 is a thin film that provides an impermeable barrier to the passage of liquids and at least some gases. In one embodiment, the backing 150 has high moisture vapor permeability, but generally impermeable to liquid water so that microbes and other contaminants are sealed out from the area under the substrate. One example of a suitable material is a high moisture vapor permeable film such as described in U.S. Pat. Nos. 3,645,835 and 4,595,001, the disclosures of which are herein incorporated by reference. In high moisture vapor permeable films or film/adhesive composites, the composite should transmit moisture vapor at a rate equal to or greater than human skin such as, for example, at a rate of at least 300 g/m²/24 hrs at 37° C./100-10% RH, or at least 700 g/m²/24 hrs at 37° C./100-10% RH, or at least 2000 g/m²/24 hrs at 37° C./100-10% RH using the inverted cup method as described in U.S. Pat. No. 4,595,001. In one embodiment, the backing 150 is an elastomeric polyurethane, polyester, or polyether block amide films. These films combine the desirable properties of resiliency, elasticity, high moisture vapor permeability, and transparency. A description of this characteristic of backing layers can be found in issued U.S. Pat. Nos. 5,088,483 and 5,160,315, the disclosures of which are hereby incorporated by reference. Commercially available examples of potentially suitable backing materials may include the thin polymer film backings sold under the tradename TEGADERM (3M Company).

Because fluids may be actively removed from the sealed environments defined by the medical dressings, a relatively high moisture vapor permeable backing may not be required. As a result, some other potentially useful backing may include, e.g., metallocene polyolefins and SBS and SIS block copolymer materials could be used.

Regardless, however, it may be desirable that the backing be kept relatively thin to, e.g., improve conformability. For example, the backing may be formed of polymer films with a thickness of 200 micrometers or less, or 100 micrometers or less, potentially 50 micrometers or less, or even 25 micrometers or less.

The adhesive 160 included on the backing 150 is typically a pressure sensitive adhesive. It is understood that the silicone article 100 may have sufficient adhesion to the backing 150 such that an adhesive 160 to secure with the silicone article 100 is unnecessary. However, if the backing 150 extends beyond the overall areas of the silicone article 100 an adhesive 160 on the backing 150 may be desirable, at least at the portions beyond the silicone article 100, to secure the backing 150 to the underlying substrate, i.e., skin.

Suitable adhesive for use on the backing include any adhesive that provides acceptable adhesion to skin and is acceptable for use on skin (e.g., the adhesive should preferably be non-irritating and non-sensitizing). Suitable adhesives are pressure sensitive and in certain embodiments have a relatively high moisture vapor transmission rate to allow for moisture evaporation. Suitable pressure sensitive adhesives include those based on acrylates, urethane, hydrogels, hydrocolloids, block copolymers, silicones, rubber based adhesives (including natural rubber, polyisoprene, polyisobutylene, butyl rubber etc.) as well as combinations of these adhesives. The adhesive component may contain tackifiers, plasticizers, rheology modifiers.

The pressure sensitive adhesives that may be used on the backing may include adhesives that are typically applied to the skin such as the acrylate copolymers described in U.S. Pat. No. RE 24,906, particularly a 97:3 isooctyl acrylate:acrylamide copolymer. Another example may include a 70:15:15 isooctyl acrylate:ethyleneoxide acrylate:acrylic acid terpolymer, as described in U.S. Pat. No. 4,737,410 (Example 31). Other potentially useful adhesives are described in U.S. Pat. Nos. 3,389,827; 4,112,213; 4,310,509; and 4,323,557.

Silicone adhesive can also be used. Generally, silicone adhesives can provide suitable adhesion to skin while gently removing from skin. Suitable silicone adhesives are disclosed in PCT Publications WO2010/056541 and WO2010/056543, the disclosure of which are herein incorporate by reference.

The pressure sensitive adhesives may, in some embodiments, transmit moisture vapor at a rate greater to or equal to that of human skin. While such a characteristic can be achieved through the selection of an appropriate adhesive, it is also contemplated that other methods of achieving a high relative rate of moisture vapor transmission may be used, such as pattern coating the adhesive on the backing, as described in U.S. Pat. No. 4,595,001. Other potentially suitable pressure sensitive adhesives may include blown-micro-fiber (BMF) adhesives such as, for example, those described in U.S. Pat. No. 6,994,904.

FIG. 3 is a bottom view of a first embodiment of a medical dressing 170 comprising a discontinuous silicone article 100, such as described in FIG. 1, and a backing 150 coated with an adhesive 160. In this embodiment, the backing 150 extends beyond the overall size of the silicone article 100 so that the adhesive 160 contacts the surface, such as skin, to further secure the medical dressing 170 to the skin. The medical dressing 170 might be positioned over a wound for the silicone article 100 to absorb wound fluid. In some instances, the silicone article 100 is placed over fragile skin to protect the skin from contact with an external surface. In some embodiments, the surface of the backing opposite the surface containing the silicone article 100 includes adhesive to secure with a device, such as a medical device.

The openings 140 are essentially free of the silicone article material, which allows for moisture vapor to pass entirely through the silicone article 100. In embodiments having a backing 150, the backing can limit the moisture vapor transmission. However, as discussed above specifically designed backing or backing/adhesive combinations can be designed to have relatively high moisture vapor transmission. In one embodiment, the silicone article 100 in combination with a backing has an moisture vapor transmission rate of at a rate of at least 300 g/m²/24 hrs at 37° C./100-10% RH, or at least 700 g/m²/24 hrs at 37° C./100-10% RH, or at least 2000 g/m²/24 hrs at 37° C./100-10% RH using the inverted cup method as described in U.S. Pat. No. 4,595,001.

The discontinuous silicone article 100 can secure to a surface. The numerous openings 140 provide flexibility, drapabality, and moisture vapor transmission away from the underlying surface. The disclosed silicone article is especially useful for contacting skin and allowing for moisture vapor transmission from the surface. In some embodiments, the discontinuous article 100 containing the silicone gel adhesive of the present disclosure are suitable for forming medical articles such as tapes, wound dressings, surgical drapes, IV site dressings, a prosthesis, an ostomy or stoma pouch, a buccal patch, or a transdermal patch.

Although specific embodiments have been shown and described herein, it is understood that these embodiments are merely illustrative of the many possible specific arrangements that can be devised. Numerous and varied other arrangements can be devised in accordance with these principles by those of ordinary skill in the art without departing from the spirit and scope of the invention. The scope of the claims should not be limited to the structures described in this application.

### EXAMPLES

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is distilled water, and all molecular weights are weight average molecular weight.

Raw materials utilized in the sample preparation are shown in Table 1.

TABLE 1
Components

| Component | Description | Supplier |
|---|---|---|
| PDMS | Xiameter ® OHX-4070, | Dow Corning |
| | hydroxyl end-capped | (Midland, MI) |
| | polydimethylsiloxane | |
| MQ | MQ 803TF Resin | Wacker Chemie AG |
| | | (Munchen, Germany) |

### Test Methods

### MVTR

The MVTR was determined with a method based on ASTM E96-80. Briefly, a 3.8 cm pattern coated silicone adhesive sample was cut and sandwiched between adhesive coated foil rings. A 118 mL glass bottle was filled with 50 mL water with a few drops of aqueous 0.2% (w/w) methylene blue. The cap for the glass bottle also contained a 3.8 cm hole. The foil ring was placed in the bottle cap and the cap was placed on the bottle with a rubber washer with a 3.6 cm opening. The bottle was placed in a 40° C., 20% relative humidity chamber in an upright position. After four hours, the bottle was removed from the chamber, sealed, and weighed (W1). The bottle was placed back in the chamber (upright position) for 24 hours at which time it was removed and reweighed (W2). The MVTR in grams of water vapor transmitted per square meter of sample area per 24 hours was calculated using the following formula.

Upright MVTR=(W1−W2)*(47,400)/24

The bottle was returned to the chamber in the upright position. After four hours, the bottle was removed from the chamber and weighed (W3). The bottle was placed back into the chamber in an inverted position for 24 hours at which time it was removed and reweighed (W4). The MVTR in grams of water vapor transmitted per square meter of sample area per 24 hours was calculated using the following formula.

Inverted MVTR=(W3−W4)*(47,400)/24

### Adhesion

Adhesion to steel was determined with a method based on ASTM D1000. Briefly, a 2.54 cm wide by 25 cm long patterned silicone adhesive sample was applied to a cleaned stainless steel plate with two passes of a 2 kg roller. An Instron tensile tester (Instron, Norwood, Mass.) was used to peel the sample at 90° at 30 cm/min. The average peel force was recorded.

### Example Formulations

A mixture of PDMS and MQ was extruded through a microprofile die, shown below, at room temperature (approximately 20° C.) onto a 25 micron, corona-treated, polyurethane film (Texin® resin, Bayer Material Science, Pittsburgh, Pa.) traveling at 9.1 m/min to produce a discontinuous silicone material. The screw in the extruder feeding the polymer strands and joining strands rotated between 45 and 105 rpm. The exit of the extruder die was approximately 4.5 cm above the polyurethane film. This discontinuous silicone material was exposed to e-beam radiation (Broadbeam EP40767, PCT Engineered Systems, LLC, Davenport, Iowa) to produce a discontinuous silicone gel adhesive. Coating weight was approximately 178 gsm (grams per square meter). Detailed conditions for the Examples are shown in Table 2.

TABLE 2
Silicone Compositions and Test Results

| | | | e-Beam | MVTR | | Adhe- |
|---|---|---|---|---|---|---|
| Exam- | Composition | Extruder | Dose | (g/m²/24 hrs) | | sion |
| ple | (w/w) | (rpm) | (MRad) | Upright | Inverted | (g/oz) |
| 1 | PDMS, 31% MQ | 90 | 7.8 | —[a] | — | 18.1 |
| 2 | PDMS, 18% MQ | 105 | 6.0 | — | — | 14.3 |
| 3 | PDMS, 18% MQ | 90 | 6.0 | — | — | 11.8 |
| 4 | PDMS, 31% MQ | 45 | 7.8 | 1132 | 1437 | — |
| 5 | PDMS, 31% MQ | 60 | 8.2 | 1123 | 1446 | — |
| 6 | PDMS, 31% MQ | 75 | 8.5 | 935 | 1176 | — |

[a]Not Tested

1. A discontinuous silicone article comprising:
a plurality of adhesive polymer strands, wherein the adhesive polymer strands comprise a radiation cured silicone gel;
a plurality of joining strands;
wherein each polymer strand repeatedly contacts an adjacent joining strand at bond regions.

2. The discontinuous silicone article of claim 1, wherein the silicone gel comprises a crosslinked poly diorganosiloxane material.

3-5. (canceled)

6. The discontinuous silicone article of claim 1, wherein the adhesive polymer strands further comprise a silicate resin tackifier.

7. The discontinuous silicone article of claim 1, wherein the adhesive polymer strands further comprise a poly(dimethylsiloxane-oxamide) linear copolymer.

8. (canceled)

9. The discontinuous silicone article of claim 2, wherein the poly diorganosiloxane material comprises a poly diorganosiloxane fluid having a dynamic viscosity at 25° C. of no greater than 1,000,000 mPa·sec.

10. The discontinuous silicone article of claim 1, wherein the adhesive polymer strands further comprise a hydrophilic polymer.

11. The discontinuous silicone article of claim 1, wherein the polymer strands and joining strands do not substantially cross over each other.

12-14. (canceled)

15. The discontinuous silicone article of claim 1, wherein the joining strands each form a substantially straight line.

16. The discontinuous silicone article of claim 1, wherein the plurality of adhesive polymer strands each form a wave.

17. The discontinuous silicone article of claim 1, further comprising an opening formed between an adhesive polymer strand and a first joining strand in an area between successive first bonding regions.

18. The discontinuous silicone article of claim 17, further comprising an opening formed between the adhesive polymer strand and a second joining strand in an area between successive second bonding regions.

19. The discontinuous silicone article of claim 18, wherein the openings form at least 25% of the area of the discontinuous silicone article.

20. (canceled)

21. The discontinuous silicone article of claim 1, wherein the joining strands are the same composition as the adhesive polymer strands.

22. The discontinuous silicone article of claim 1, further comprising:
a backing to which the plurality of polymer strands and joining strands are secured.

23-26. (canceled)

27. A method of making a discontinuous silicone article comprising:
dispensing through a first orifice at a first speed a polymer strand, which comprises silicone material;
dispensing through a second orifice at a second speed a first joining strand on a first side of the polymer strand, wherein the first speed is faster than the second speed;
dispensing through a third orifice at the second speed a second joining strand on a second side of the polymer strand, opposite the first side;
applying radiation to the silicone material to cure the silicone material to form a silicone gel.

28. (canceled)

29. The method of making a discontinuous silicone article of claim 27, wherein the first joining strand and second joining strand are the same composition as the polymer strands.

30-31. (canceled)

32. The method of making a discontinuous silicone article of claim 27, further comprising:
oscillating the polymer strand between the first joining strand to form a first bond region and the second joining strand to form a second bond region.

33-34. (canceled)

35. The method of making a discontinuous silicone article of claim 32, further comprising:
forming an opening between the polymer strand and the first joining strand in an area between the successive first bonding regions.

36. The method of making a discontinuous silicone article of claim 35, further comprising:
forming an opening between the polymer strand and the second joining strand in an area between the successive second bonding regions.

37. The method of making a discontinuous silicone article of claim 27, further comprising:
applying e-beam radiation to the silicone material to cure the silicone material to form a silicone gel.

38-40. (canceled)
